(19) United States
(12) Patent Application Publication
(10) Pub. No.: US 2022/0102010 A1
(43) Pub. Date: Mar. 31, 2022

(54) SYSTEMS AND METHODS FOR MODELLING A HUMAN SUBJECT

(71) Applicant: KONINKLIJKE PHILIPS N.V., Eindhoven (NL)

(72) Inventors: Cornelis Petrus HENDRIKS, Eindhoven (NL); Murtaza Bulut, Eindhoven (NL); Lieke Gertruda Elisabeth COX, Eindhoven (NL); Valentina LAVEZZO, Eindhoven (NL)

(21) Appl. No.: 17/353,949

(22) Filed: Jun. 22, 2021

Related U.S. Application Data
(60) Provisional application No. 63/083,198, filed on Sep. 25, 2020.

(51) Int. Cl. G16H 50/50; G06F 3/0484; A61B 5/00; G16H 50/70; G16H 50/20; G16H 40/67

(52) U.S. Cl. CPC G16H 50/50; G06F 3/0484; A61B 5/7275; A61B 5/7264; A61B 5/6801; A61B 34/10; A61B 5/0031; G16H 50/70; G16H 50/20; G16H 40/67; A61B 5/4842; A61B 5/6898

(57) ABSTRACT

Proposed are methods and systems for generating a prediction for a first organ of a subject using a Digital Twin model of a second, different organ of the subject. By obtaining an understanding of a relationship between the first and second organ, a Digital Twin prediction for the second organ is used to determine a prediction for the first organ

### FIELD OF THE INVENTION

The invention relates to modelling human subjects, and more particularly to the field of subject-specific models of biological function (commonly referred to as digital twins).

### BACKGROUND OF THE INVENTION

A recent development in healthcare is the so-called ‘Digital Twin’ concept. In this concept, a digital representation or computational simulation (i.e. the digital twin model) of a physical system is provided and connected to its physical counterpart, for example through the Internet of things as explained in US 2017/286572 A1 for example. Through this connection, the Digital Twin (DT) model typically receives data pertaining to the state of the physical system, such as sensor readings or the like, based on which the DT model can predict the actual or future status of the physical system, e.g. through simulation.

Such DT technology is also becoming of interest in the medical field, as it provides an approach to more efficient medical care provision. For example, a DT model may be built using imaging data of a subject (i.e. patient), e.g. a person suffering from a diagnosed medical condition as captured in the imaging data.

A DT model of a subject (i.e. a subject-specific computational simulation) may serve a number of purposes. Firstly, the DT model (rather than the patient) may be subjected to a number of virtual tests, e.g. treatment plans, to determine which treatment plan is most likely to be successful to the patient. This reduces the number of tests that physically need to be performed on the actual patient. Secondly, the DT model of a subject may be used to predict the onset, treatment (outcome) or development of medical conditions of the subject. That is, the DT model of a subject may offer a healthcare professionals advanced visualization and/or physical insights into health information of the subject, thus supporting improved Clinical Decision Support (CDS).

However, a DT model of an organ may not always be available. This may be because an organ DT model is not available for a specific subject, or because an organ DT model is not available in general (e.g. because the technology has not yet been developed). Consequently, when a problem or disease manifests itself in an organ and impairs the function of that organ, a healthcare professional or clinician may not investigate and/or manage the problem/disease with a DT model of that organ.

### SUMMARY OF THE INVENTION

The invention is defined by the claims.

According to examples in accordance with an aspect of the invention, there is provided a system for generating a prediction for a first organ of a subject, the system comprising:

an input interface configured to obtain: first input data representative of a detected property of the first organ of the subject; and second input data representative of a detected property of a second, different organ of the subject;

an data analysis component configured to determine a relationship between the first and second input data; and

a processor arrangement configured to:

evaluate the second input data with a digital twin of the second organ to generate a prediction for the second organ; and

generate a prediction for the first organ based on the determined relationship and the prediction for the second organ.

Embodiments propose concepts for making predictions about a first organ based on DT model predictions for a second organ. Such proposals are based on a realization that organ interactions may provide opportunities for using DT models of other organs in making predictions for an organ. Organ interactions may be identified based on analysis of physiological signals measured by sensors (such as wearable sensors or subject monitors).

As a result, systems and methods are proposed for predicting a disease progression, future parameter value, future status, or a function evolution of a first organ, based on DT model predictions for a second organ. Put another way, the future (e.g. a future physiological property value or future status) of a first organ may be predicted without using or having a DT model of the first organ. To obtain a prediction for a first organ, embodiments may leverage DT models of one or more other organs, thus avoiding the need for a DT model of the first organ.

By using an available DT model for another organ, and using an understanding of a relationship between that other organ and a target organ, predictions made by the DT model can be translated or converted to predictions for the target organ.

A disease and its impact is often not restricted to a single organ, because often diseases are systemic and organs interact. That is, in a human, multiple organs work together in biological systems (respiratory, cardiovascular, digestive system etc.). Communication and interaction takes place via physical, neurological, endocrine, biochemical and immunological pathways. Similarly, in a DT model of a subject, organs of different type and nature work together in sub-systems (organ systems) and a super-system (human body). It is therefore proposed that organ interaction offers an opportunity to use DT models of other organs in the disease prediction and management of a first (i.e. target) organ.

Embodiments may therefore be of particular use for supporting clinical decision making. Exemplary usage applications may for example, relate to predicting the onset, treatment (outcome) or development of medical conditions and/or medical procedures. Embodiments may thus be of particular use in relation to medical care management and/or prediction.

In an embodiment, the data analysis component may comprise a correlation component configured to determine a measure of correlation between the first and second organs based on the first and second input data. The data analysis component may then be configured to compare the determined measure of correlation between the first and second organs against a requirement and to determine a correlation function representative of a mapping (i.e. representation of a relationship) between the first and second input data based on the comparison result. Some embodiments may therefore employ a concept of checking the level of correlation between organs, and then making the determination of the relationship or correlation function dependent on the level of correlation being above a required/minimum threshold level. In this way, processing resource and requirements may be minimized (through the avoidance of unwarranted/unnecessary analysis). Further, the requirement may be predetermined and/or modified according to particular needs or preferences. For instance, the requirement may be a threshold value that must be met or exceeded, and the threshold value may be user-defined and/or adapted/modified dynamically.

Further, the correlation component may be configured to determine an inner product of the first and second input data. Relatively simple and/or well-known mathematical operations and calculations may therefore be employed by embodiments, thus minimizing the cost and/or complexity associated with implementations.

In some embodiments, the input interface may be configured to obtain third input data representative of a detected property of a third organ of the subject. The correlation component may then be further configured to determine a measure of correlation between the first and third organ based on the first and third input data, and the data analysis component may be configured to determine the requirement based on the determined measure of correlation between the first and third organs. In this way, embodiments may consider a third organ, and then set the requirement (e.g. threshold value) in consideration of the correlation between the first and third organ. Embodiments may therefore employ additional DT models and thus improve prediction accuracy.

Further, the data analysis component may be configured to determine the requirement to be that the measure of correlation between the first and second organs must exceed the measure of correlation between the first and third organs. That is, a measure of correlation between the first and third organs may define a minimum value that the measure of correlation between the first and second organs is required to exceed The data analysis component may then be configured to determine a correlation function representative of a mapping between the first and second input data responsive to the requirement being met. In other words, ‘coupling’ of the first organ to the second and third organs may be compared and then the second organ only proceeded with if the first organ's coupling with the second organ is at a certain level compared to its coupling with the third organ (e.g. if coupling between the first and second organs is greater than coupling between the first and third organs).

In an embodiment, determining the relationship between the first and second input data may comprise processing the first and second input data with at least one of: a parametric method; and a neural network. Various methods to determine the correlation function may therefore be employed. In this way, embodiments may be configured to leverage various forms of information to improve prediction accuracy.

In some embodiments, the processor arrangement may be configured to generate a prediction for the first organ further based on a measure of variability of the first and second input data. A concept of variability analysis may thus be employed when generating the prediction for the first organ. In this way, improved prediction accuracy may be achieved.

The input interface may be further configured to obtain supplementary input data representative of a detected property of the first organ of the subject at the future point in time. The data analysis component may then be configured to determine an accuracy of the determined relationship based on the supplementary input data. Embodiments may thus employ a concept of obtaining data for a future point in time and then using that data to assess the accuracy of the originally determine relationship (e.g. accuracy of an originally determined correlation function). Further, the data analysis component may be further configured to modify the determined relationship based on the supplementary input data. Feedback concepts may thus be employed for improved accuracy and/or continued improvement of embodiments.

Some embodiments may further comprise at least one of: a first set of one or more sensors configured to detect a property of the first organ of the subject and to generate first input data representative of the detected property of the first organ of the subject; and a second set of one or more sensors configured to detect a property of the second organ of the subject and to generate second input data representative of the detected property of the second organ of the subject. Embodiments may thus include one or more sensors for obtaining organ data. By way of example, at least one of the first or second sets of one or more sensors may comprise: a wearable sensor; a mobile phone; a digital imaging device; or a body implant. Embodiments may therefore leverage various forms of device and/or information to improve prediction accuracy.

In an embodiment, the processor arrangement may be further configured to generate a control instruction for a sensor or medical equipment based on the prediction for the first organ. In this way, a sensor and/or medical equipment may be controlled according to results (e.g. predictions) generated by embodiments. Dynamic and/or automated control concepts may therefore be realized by proposed embodiments.

Further, proposed concepts may provide a clinical decision support comprising a system according to a proposed embodiment.

According to examples in accordance with another aspect of the invention, there is provided a method for generating a prediction for a first organ of a subject. The method comprises:

obtaining: first input data representative of a detected property of the first organ of the subject; and second input data representative of a detected property of a second, different organ of the subject;

determining a relationship between the first and second input data; and

evaluating the second input data with a digital twin of the second organ to generate a prediction for the second organ; and

generating a prediction for the first organ based on the determined relationship and the prediction for the second organ.

According to examples in accordance with yet another aspect of the invention, there is provided a computer program product comprising computer program code means which, when executed on a computing device having a processing system, cause the processing system to perform all of the steps of the method described above.

These and other aspects of the invention will be apparent from and elucidated with reference to the embodiment(s) described hereinafter.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is simplified block diagram of a system according to the exemplary embodiment;

FIG. 2 is a graph depicting the exemplary variation of an organ property value for the first and second organs of FIG. 1 with respect to time;

FIG. 3 depicts an exemplary embodiment for predicting a future status of a first organ of a subject, wherein the process(es) undertaken in various different elements of the system are separately illustrated;

FIG. 4 is a flow diagram of a method for generating a prediction for a first organ of a subject according to an embodiment;

FIG. 5 is a simplified block diagram of a system for generating a prediction for a first organ of a subject according to an embodiment; and

FIG. 6 illustrates an example of a computer within which one or more parts of an embodiment may be employed.

### DETAILED DESCRIPTION OF THE EMBODIMENTS

The invention will be described with reference to the Figures.

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the Figures to indicate the same or similar parts.

The invention provides concepts for generating a prediction for a first organ of a subject using a DT model of a second, different organ of the subject. By obtaining an understanding of a coupling (or relation/correlation) between the first and second organ, a prediction for the second organ may be converted (i.e. mapped or translated) to a prediction for the first organ.

Proposed embodiments may therefore leverage a DT model for a second organ when a DT model for a first organ is not available. In this way, existing DT models may be used in new ways so as to provide improved understanding and/or predictions for organs that do not currently have DT models.

Embodiments may enable more accurate modeling of the disease progression, physiological parameter changes, and/or future disease state of a subject and their need for imminent medical intervention, especially where a DT model for an organ it not available but a DT model for another organ is available. Embodiments may therefore be of particular use for supporting clinical decision-making. For instance, predictions for a first organ (obtained based on DT model predictions for a second organ) may be used in clinical decision support and chronic disease management.

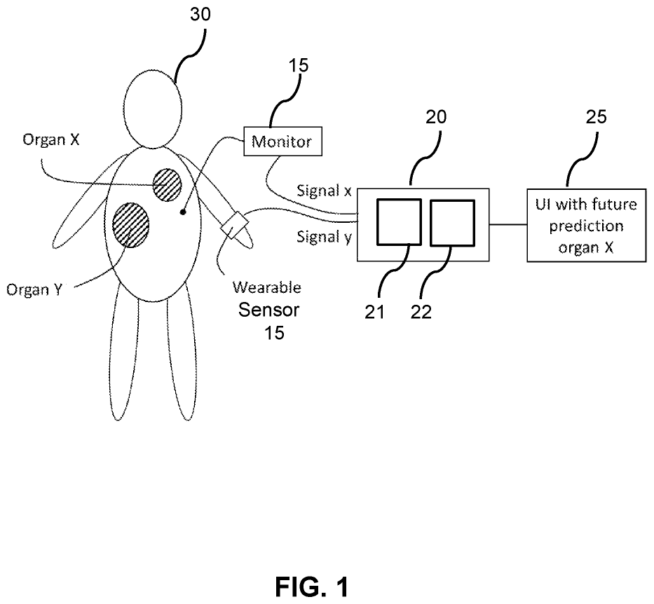

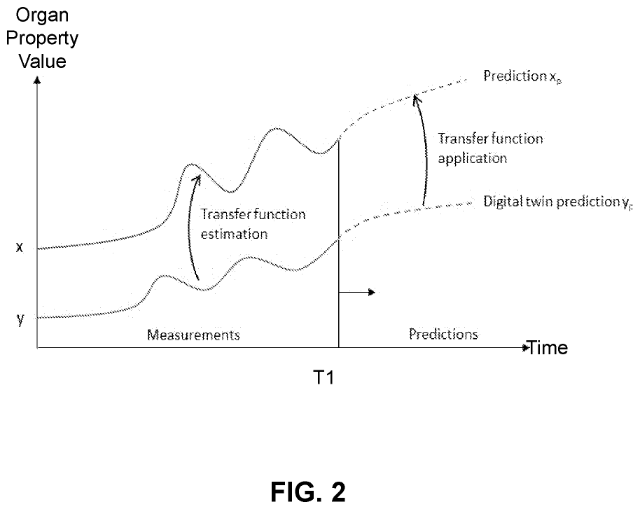

Referring to FIGS. 1 and 2, there is illustrated an exemplary embodiment,

FIG. 1 is simplified block diagram of a system according to the exemplary embodiment, and FIG. 2 is a graph depicting the exemplary variation of an organ property value for the first and second organs with respect to time. In FIG. 2, a measurement signal x (uppermost line) is derived from the first organ X, and a measurement signal y (lower line) is derived from organ Y. Accordingly, the signals x and y may be considered as representing a (disease) state or stage of the organ directly (or indirectly), for example a low blood oxygenation in the case of COPD. In the latter case, a low oxygenation can be caused by COPD, but it does not represent the COPD stage. For example, the COPD stage might be constant while the blood oxygenation fluctuates with exacerbations. Both the disease state or stage, and the derived signal may be valuable for a clinician.

The system comprises subject sensors 15A, 15B for detecting one or more properties of an organ.

Here it is noted that reference to a ‘property of an organ’ should be taken to refer to any one of a range of attributes, qualities, characteristics, traits of features of an organ that may be measured, sensed, detected or otherwise determined. Purely by way of example only, properties of an organ may include: a disease state; a physical property or characteristic (such as size/dimension, colour, texture weight, etc.); a physiological parameter; or a function measure. Also, detection of a property of an organ need not be limited to being undertaken by a sensor, and may instead be undertaken using any suitable means for determining a value of a property (including human assessment).

The system also comprises a computing system 20 (with data analysis component 21 and processor arrangement 22), and a user interface 25 for a clinician. The system is configured to predict the progression of a first organ X of the subject 30 based on a DT model of a second organ Y of the subject 30.

Based on analysis of the signals x and y up until a current time Ti, a correlation function representative of a relationship between the signals x and y may be determined. That is, as illustrated in FIG. 2, a transfer function estimation for mapping from the signal y for the second organ to signal x for the first organ may be obtained. Such analysis and correlation determination can be undertaken using various known techniques, including methods provided by Bartsch (Bartsch R P, Liu K K L, Bashan A, Ivanov P C. (2015) Network Physiology: How Organ Systems Dynamically Interact. PLoS ONE 10(11): e0142143. doi:10.1371/journal.pone.0142143) or Joshi (Joshi, R. (2019). Towards automated solutions for predictive monitoring of neonates. Eindhoven: Technische Universiteit Eindhoven). Alternatively, a relationship can be explicitly described or indicated by a medical professional via the user interface 25.

Subsequently, a future prediction yₚ for the second organ Y (obtained from the DT model of the second organ) may be mapped or converted to a prediction xₚ for the first organ X, via application of the estimated transfer function (i.e. determined correlation function). In this way, the future progression of the first organ X (after current time Ti) may be predicted using: (i) a DT model of the second organ Y; and (ii) the estimated transfer/correlation function representing a relationship between the signals x and y.

The prediction can be done at one point in time, or continuously. Also, the transfer/correlation function may be repeatedly or continuously updated as more data becomes available over time.

Although not illustrated, a correlation function representative of a relationship between the first x and second y organ signals may be derived based on multiple signals, or from the individual measurements or data points.

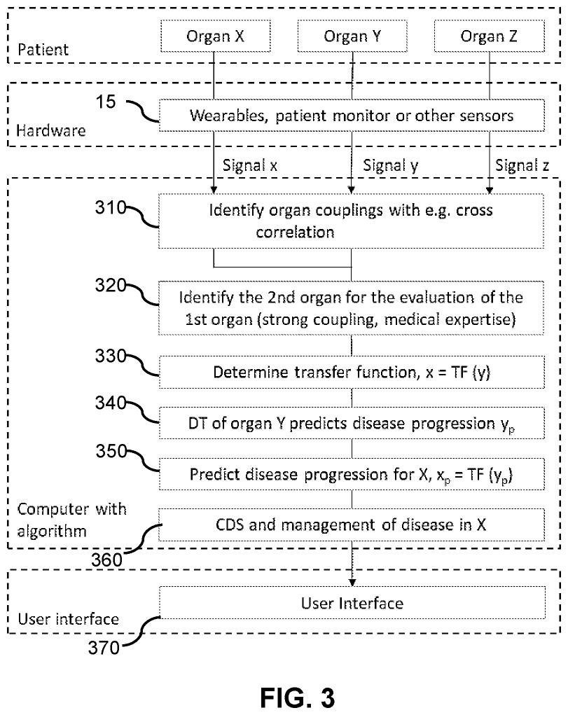

By way of further illustration of the proposed concept(s), an exemplary embodiment will now be described with reference to FIG. 3.

FIG. 3 illustrates an exemplary embodiment for predicting a future status of a first organ of a subject, wherein the process(es) undertaken in various different elements of the system are separately illustrated.

The starting point is that a first organ X is diseased and needs treatment, but a digital twin of that organ is not available. The system implement a proposed method to predict a disease progression, or a function evolution, of the first organ X when a digital twin model for the first organ X is not available. The signals represent or characterize organ function or dis-function, i.e. a disease.

Signals x, y, z are obtained from sensors implemented in hardware such as wearable devices, patient monitors, wired electrodes, mobile phones, camera's, implantables etc. The signals x, y, z from these devices represent organ functions, dis-functions (diseases) or vital signs (i.e. various organ properties). The sources of these signals x, y, z are first to third organs X, Y, Z, respectively. Although, in this exemplary embodiment, only a single signal for a single organ is obtained from each sensor, it is to be understood that signals for multiple organs may be obtained from a single sensor. For example, a PPG sensor which measures heart rate, blood pressure and respiration rate.

As explained above, Bartsch (Bartsch R P, Liu K K L, Bashan A, Ivanov P C. (2015) Network Physiology: How Organ Systems Dynamically Interact. PLoS ONE 10(11): e0142143. doi:10.1371/journal.pone.0142143) describes how to determine couplings (i.e. relationships) between organs from cross correlations between signals (step 310). Cross correlation is a mathematical signal processing technique to determine the similarity or association between two signals x(t) and y(t). Cross correlation calculates a sliding inner product of two or more signals, which provides insight in the similarity as a function of a time lag τ applied to one of the signals, R=x\textcircled{x}y. Normalization by extracting the mean signal values and dividing by the standard deviation of the signals provides a scale-free measure for the strength of the correlation, i.e. the Pearson correlation coefficient p, and or the Spearman coefficient r. The value of the Pearson coefficient, which lies between −1>ρ>1, can be used to interpret the strength of the correlation in a quantitative manner. A consistent time lag τ=T indicates an association between two signals. The Spearman coefficient assesses how well the relationship between two variables can be described using a monotonic function.

Besides the method of Bartsch (Bartsch R P, Liu K K L, Bashan A, Ivanov P C. (2015) Network Physiology: How Organ Systems Dynamically Interact. PLoS ONE 10(11): e0142143. doi:10.1371/journal.pone.0142143), there are other signal processing techniques to identify couplings, for example phase-rectified signal averaging (PRSA) and bivariate phase rectified signal averaging (BPRSA) applied by Joshi (Joshi, R. (2019). Towards automated solutions for predictive monitoring of neonates. Eindhoven: Technische Universiteit Eindhoven) to determine cardiorespiratory coupling.

After identifying organ couplings (step 310), it is proposed to identify (step 320) the organ that has a Digital Twin for the evaluation of the disease in the first organ X.

The organ which is a candidate for the evaluation of the disease progression in organ X, can be selected from the results in step 310. For example, signals with a strong coupling, e.g. a high Pearson or Spearman coefficient, or a consistent time lag, indicate organs which are coupled most with the first organ X. Here, it is noted that a strong coupling may not necessarily mean that a second organ Y is the root cause of a disease in organ X. However, the second organ Y being a root cause may not be required. Rather, a strong coupling between first X and second Y organs may be is sufficient for determining a correlation between the first X and second Y organs. Alternatively, a strong coupling and thereby a selection of the second organ Y, can be determined from prior medical expertise (e.g. a diagnosed comorbidity).

Next, in step 330, a correlation function (i.e. transfer function) representing relationship between x and y is determined. Estimation of a correlation function between two signals is well known, and this includes parametric methods (e.g. linear prediction) and data driven methods (e.g. using convolutional neural networks, and deep learning in general). In the case of a linear time invariant system with a single input and a single output (as indicated, for example, by the cross correlation results), a linear regression may be sufficient. In more complicated systems, alternative methods are available. Such methods are well known, and so are omitted from this description. As a result of determining a correlation function, an understanding of a relation between a disease process in the first organ X and a disease process in the second organ Y is obtained from the correlation function x=TF(y). This correlation function can be a parametric or non-parametric.

Digital Twin prediction for the second organ Y is then undertaken in step 340. That is, the digital twin model of the second organ Y is used to predict the disease progression of the second organ Y. By way of example, in this embodiment, the output of digital twin model of the second organ Y is a predicted time series yₚ.

The disease progression in the first organ X is then predicted (in step 350) based on the output of prediction for the second organ Y (from step 340). Specifically, once the digital twin prediction y=yₚ is known, the prediction for signal x can be determined with using the correlation function, xₚ=TF(yₚ). A variability analysis may also be undertaken, e.g. a Monte Carlo analysis to evaluate the spread in the output signal for organ X and Y.

Clinical decision support (Step 360) may then be provided bases on the prediction for the first organ X. For example, the disease progression prediction for the first organ X can be used in several ways, such as: initiating monitoring; scheduling extra diagnostic tests; provide early warning(s); predict and decide on the timing of interventions such (surgery, medication etc.), or predict the progression from one disease stage to the next.

A user interface 370 (e.g. display or signal output component) is provided in this example to communicate the recommended action to the user. In some embodiments, the process of determining the correlation function may be repeated (e.g. to account for changes). For instance, there may be cases where the mapping between the first and second input data changes as a result of an implemented medical treatment or change in patient condition. In such a case, the correlation function may be determined again in order to maintain and/or improve its accuracy.

By way of yet further illustration of the proposed concept(s), an exemplary use case will now be described wherein there is heart-lung coupling with Atrial Fibrillation (AF) (i.e. COPD interaction).

The root cause of atrial fibrillation (AF) is typically a secondary medical condition such as hypertension, heart failure, valvular heart disease, myocardial infarction, thyroid dysfunction, obesity, diabetes, chronic obstructive pulmonary disease, chronic kidney disease, etc. The identification of such conditions, and their prevention and treatment is an important leverage to prevent AF and its disease burden. Knowledge of these secondary conditions and their management is therefore important for optimal management of AF patient.

By way of example, COPD and its severity is a predictor for AF progression, treatment outcomes and mortality. The strong link between COPD and AF is based on a number of pathophysiological mechanisms such as hypoxia, hypercapnia, pulmonary hypertension, ventricular adaptation, inflammation, oxidative stress, and respiratory drugs. Clinically, strong links exists between COPD and AF. For example, the lung function parameter FEV1 (Forced Expiration Volume in 1 second) is inversely correlated with the incidence of AF, the presence of AF has a significant influence on mortality especially in COPD exacerbations, COPD exacerbations predict incident AF, and COPD (HATCH score) is related to stroke risk.

Wearable devices that measure a blood pulse signal (electrically, optically or mechanically) are suitable for acquiring longitudinal AF data. Spirometry (a lung function test) is current the standard approach for monitoring the severity and progression of COPD. However, remote and wearable monitoring technologies are being introduced. As an example, a PPG sensor integrated in a wearable provides information on blood pulse and blood oxygenation. Thus, a single sensor can obtain two signals to characterize two diseases (AF and COPD).

Using such sensors and devices, the longitudinal monitoring of AF signs (e.g. irregular fast heart beats) can be undertaken. For example, a short-term feature is an irregular fast heart-beat. A long-term feature is, for instance, the amount of the time the subject is in an AF episode per time period (e.g. per week, month or year). Further, multiple physiological signals are available to monitor COPD, for example blood oxygenation, cardiac electrical activity (ECG), respiratory rate, blood pressure, spirometry, etc.

A known digital twin model of long-term emphysema progression in the lung or a known short-term exacerbation risk prediction model can be used to predict the progression of COPD signals.

The AF disease progression prediction can be used in several ways. For example, by starting ECG monitoring in a timely manner, the predicted speed of progression may provide as an early warning for stroke, and/or may define the timing of interventions such as ablation. Generated predictions may also test the impact of a COPD treatment (such as the dosing of respiratory drugs) or predict the progression from one AF stage to the next.

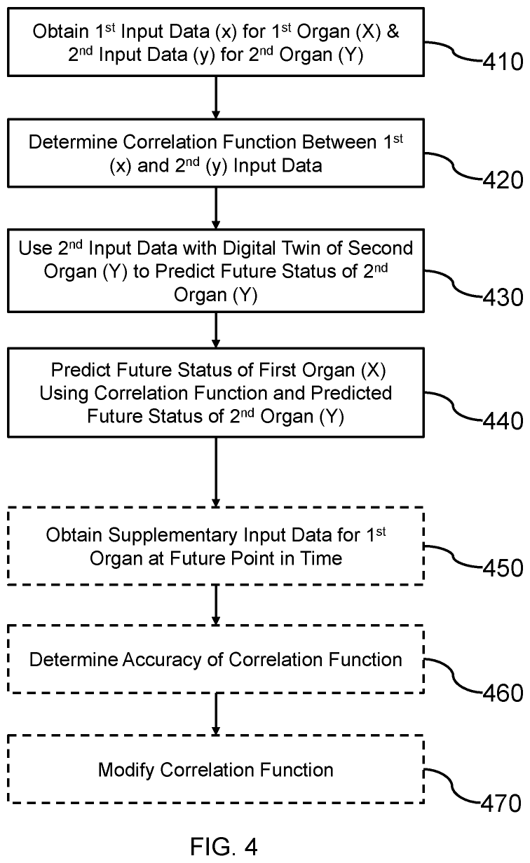

Referring now to FIG. 4, there is depicted a flow diagram of a method for generating a prediction for a first organ of a subject according to an embodiment.

The method begins with step 410 of obtaining (410): first input data (x) representative of a detected property of the first organ (X) of the subject; and second input data (y) representative of a detected property of a second, different organ of the subject.

Next, in step 420, a relationship between the first and second input data is determined. Here, the relationship is defined by a correlation function representative of a mapping between the first and second input data.

In step 430, the second input data is evaluated with a digital twin of the second organ to generate a prediction of a future status of the second organ.

Then, in step 440, a prediction for the first organ is determined based on the determined relationship and the prediction for the second organ.

By way of illustrating how the determined relationship may be modified or updated (e.g. to account for changes over time), the method may also comprise additional steps (as indicated by the dashed boxes). Specifically, the exemplary method of FIG. 4 may include additional steps 450, 460 and 470.

Step 450 comprises obtaining supplementary input data representative of a detected property of the first organ of the subject at the future point in time. Step 460 then comprises determining an accuracy of the relationship based on the supplementary input data. Based on the supplementary input data and/or the determined accuracy of the relationship, the relationship is then modified (i.e. re-calculated) in step 470.

Although the exemplary method of FIG. 4 is configured to predict the progression of the first organ X based on a digital twin model of one other organ, it should be understood that other embodiment may use digital twins for more than one other organ. For example, proposed concepts may predict the progression of the first organ X based on digital twin models for second Y and third Z organs. This may, for example, be employed for cases where the first organ X is strongly coupled with more than one other organ.

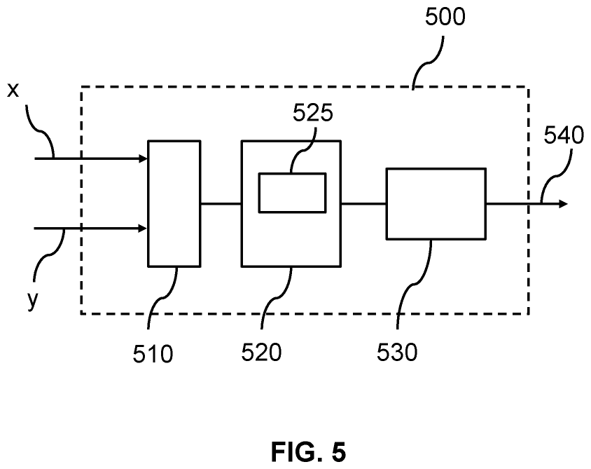

Referring now to FIG. 5, there is illustrated a simplified block diagram of a system 500 for generating a prediction for a first organ of a subject according to an embodiment. The system comprises an input interface 510 that is configured to obtain: first input data x representative of a detected property (e.g. physical property value, disease state/measure, etc.) of the first organ X of the subject; and second input data y representative of a detected property of a second, different organ Y of the subject. In this example, the input interface 510 comprises any suitable communication interface adapted to receive first x and second y input data signals from respective sensor devices.

The input interface 510 provides the first x and second y input data to a data analysis component 520 of the system 500. The data analysis component 510 is configured to determine a correlation function representative of a relationship between the first x and second y input data. Here, determining the correlation function comprises processing the first x and second y input data with a neural network. That is, the data analysis component 520 comprises a neural network that is adapted to predict/calculate the correlation function.

Specifically, the data analysis component 520 comprise a correlation component 525 (employing a neural network) that is configured to determine a measure of correlation between the first X and second Y organs based on the first x and second y input data. Further, the data analysis component 525 is configured to compare the determined measure of correlation between the first and second organs against a requirement (e.g. threshold value) and to determine a correlation function representative of a mapping between the first and second input data based on the comparison result. For instance, the data analysis component 525 may only determine a correlation function if the measure of correlation exceeds a minimum required value (e.g. represented by a threshold value).

The system also comprises a processor arrangement 530 that is configured to evaluate the second input data with a digital twin model of the second organ to generate a prediction of a future status of the second organ. Based on the determined correlation function and the prediction of a future status of the second organ at future point, the processor arrangement then generates a prediction of a future status of the first organ. In doing so, the processor arrangement may generate the prediction in consideration of measure of variability of the first and second input data. That is, the system may be adapted to account for reliability or consistency characteristics of the input data.

Although the system 500 has been described as only employing first x and second y input data from first X and second Y organs, respectively, other implementations of the system may employ data from three or more organs.

For example, in an alternative implementation of the system 500, the input interface 510 is configured to obtain third input data representative z of a detected property of a third organ Z of the subject. The correlation component 525 is then further configured to determine a measure of correlation between the first X and third Z organ based on the first x and third z input data. Based on the determined measure of correlation between the first X and third Z organs, the data analysis (520) component is configured to determine the requirement (e.g. threshold value) to be that the measure of correlation between the first X and second Y organs must exceed the measure of correlation between the first X and third Z organs. In this way, the data analysis component 520 can then be configured to only determine a correlation function representative of a mapping between the first x and second y input data responsive to the requirement being met.

That is, a correlation function mapping between the first x and second y input data is only determined if the measure of correlation (i.e. coupling) between the first X and second Y organs is greater than measure of correlation between the first X and third Z organs. If it is not, a correlation function mapping between the first x and third z input data is determined instead. This approach ensures that the correlation function is determined for the strongest coupling to the first organ X. Put another way, the ‘coupling’ of the first organ to the second and third organs may be compared and then the second organ only proceeded with if the first organ's coupling with the second organ is greater than coupling between the first and third organs.

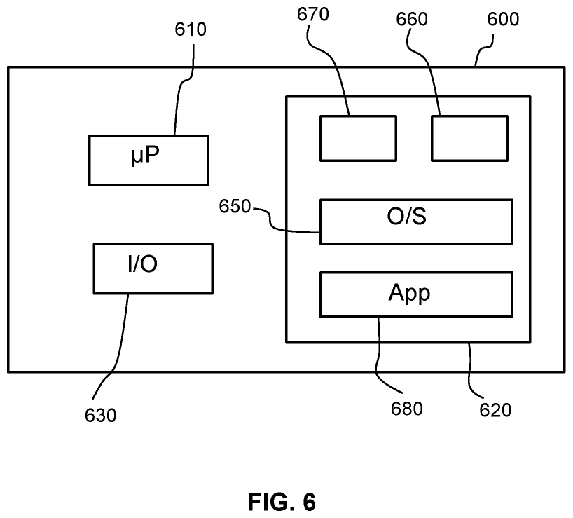

By way of further example, FIG. 6 illustrates an example of a computer 600 within which one or more parts of an embodiment may be employed. Various operations discussed above may utilize the capabilities of the computer 600. For example, one or more parts of a system for calculating an expiration time of a DT model for a subject may be incorporated in any element, module, application, and/or component discussed herein. In this regard, it is to be understood that system functional blocks can run on a single computer or may be distributed over several computers and locations (e.g. connected via internet).

The computer 600 includes, but is not limited to, PCs, workstations, laptops, PDAs, palm devices, servers, storages, and the like. Generally, in terms of hardware architecture, the computer 600 may include one or more processors 610, memory 620, and one or more I/O devices 630 that are communicatively coupled via a local interface (not shown). The local interface can be, for example but not limited to, one or more buses or other wired or wireless connections, as is known in the art. The local interface may have additional elements, such as controllers, buffers (caches), drivers, repeaters, and receivers, to enable communications. Further, the local interface may include address, control, and/or data connections to enable appropriate communications among the aforementioned components.

The processor 610 is a hardware device for executing software that can be stored in the memory 620. The processor 610 can be virtually any custom made or commercially available processor, a central processing unit (CPU), a digital signal processor (DSP), or an auxiliary processor among several processors associated with the computer 600, and the processor 610 may be a semiconductor based microprocessor (in the form of a microchip) or a microprocessor.

The memory 620 can include any one or combination of volatile memory elements (e.g., random access memory (RAM), such as dynamic random access memory (DRAM), static random access memory (SRAM), etc.) and non-volatile memory elements (e.g., ROM, erasable programmable read only memory (EPROM), electronically erasable programmable read only memory (EEPROM), programmable read only memory (PROM), tape, compact disc read only memory (CD-ROM), disk, diskette, cartridge, cassette or the like, etc.). Moreover, the memory 620 may incorporate electronic, magnetic, optical, and/or other types of storage media. Note that the memory 620 can have a distributed architecture, where various components are situated remote from one another, but can be accessed by the processor 610.

The software in the memory 620 may include one or more separate programs, each of which comprises an ordered listing of executable instructions for implementing logical functions. The software in the memory 620 includes a suitable operating system (0/S) 650, compiler 660, source code 670, and one or more applications 680 in accordance with exemplary embodiments. As illustrated, the application 680 comprises numerous functional components for implementing the features and operations of the exemplary embodiments. The application 680 of the computer 600 may represent various applications, computational units, logic, functional units, processes, operations, virtual entities, and/or modules in accordance with exemplary embodiments, but the application 680 is not meant to be a limitation.

The operating system 650 controls the execution of other computer programs, and provides scheduling, input-output control, file and data management, memory management, and communication control and related services. It is contemplated by the inventors that the application 680 for implementing exemplary embodiments may be applicable on all commercially available operating systems.

Application 680 may be a source program, executable program (object code), script, or any other entity comprising a set of instructions to be performed. When a source program, then the program is usually translated via a compiler (such as the compiler 660), assembler, interpreter, or the like, which may or may not be included within the memory 620, so as to operate properly in connection with the O/S 650. Furthermore, the application 680 can be written as an object oriented programming language, which has classes of data and methods, or a procedure programming language, which has routines, subroutines, and/or functions, for example but not limited to, C, C++, C#, Pascal, BASIC, API calls, HTML, XHTML, XML, ASP scripts, JavaScript, FORTRAN, COBOL, Perl, Java, ADA, .NET, and the like.

The I/O devices 630 may include input devices such as, for example but not limited to, a mouse, keyboard, scanner, microphone, camera, etc. Furthermore, the I/O devices 630 may also include output devices, for example but not limited to a printer, display, etc. Finally, the I/O devices 630 may further include devices that communicate both inputs and outputs, for instance but not limited to, a NIC or modulator/demodulator (for accessing remote devices, other files, devices, systems, or a network), a radio frequency (RF) or other transceiver, a telephonic interface, a bridge, a router, etc. The I/O devices 630 also include components for communicating over various networks, such as the Internet or intranet.

If the computer 600 is a PC, workstation, intelligent device or the like, the software in the memory 620 may further include a basic input output system (BIOS) (omitted for simplicity). The BIOS is a set of essential software routines that initialize and test hardware at startup, start the 0/S 650, and support the transfer of data among the hardware devices. The BIOS is stored in some type of read-only-memory, such as ROM, PROM, EPROM, EEPROM or the like, so that the BIOS can be executed when the computer 600 is activated.

When the computer 600 is in operation, the processor 610 is configured to execute software stored within the memory 620, to communicate data to and from the memory 620, and to generally control operations of the computer 300 pursuant to the software. The application 680 and the 0/S 650 are read, in whole or in part, by the processor 310, perhaps buffered within the processor 610, and then executed.

When the application 680 is implemented in software it should be noted that the application 680 can be stored on virtually any computer readable medium for use by or in connection with any computer related system or method. In the context of this document, a computer readable medium may be an electronic, magnetic, optical, or other physical device or means that can contain or store a computer program for use by or in connection with a computer related system or method.

The application 680 can be embodied in any computer-readable medium for use by or in connection with an instruction execution system, apparatus, or device, such as a computer-based system, processor-containing system, or other system that can fetch the instructions from the instruction execution system, apparatus, or device and execute the instructions. In the context of this document, a “computer-readable medium” can be any means that can store, communicate, propagate, or transport the program for use by or in connection with the instruction execution system, apparatus, or device. The computer readable medium can be, for example but not limited to, an electronic, magnetic, optical, electromagnetic, infrared, or semiconductor system, apparatus, device, or propagation medium.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

A single processor or other unit may fulfill the functions of several items recited in the claims.

It will be understood that the disclosed methods are computer-implemented methods. As such, there is also proposed a concept of a computer program comprising code means for implementing any described method when said program is run on a processing system.

The skilled person would be readily capable of developing a processor for carrying out any herein described method. Thus, each step of a flow chart may represent a different action performed by a processor, and may be performed by a respective module of the processing processor.

As discussed above, the system makes use of a processor to perform the data processing. The processor can be implemented in numerous ways, with software and/or hardware, to perform the various functions required. The processor typically employs one or more microprocessors that may be programmed using software (e.g. microcode) to perform the required functions. The processor may be implemented as a combination of dedicated hardware to perform some functions and one or more programmed microprocessors and associated circuitry to perform other functions.

Examples of circuitry that may be employed in various embodiments of the present disclosure include, but are not limited to, conventional microprocessors, application specific integrated circuits (ASICs), and field-programmable gate arrays (FPGAs).

In various implementations, the processor may be associated with one or more storage media such as volatile and non-volatile computer memory such as RAM, PROM, EPROM, and EEPROM. The storage media may be encoded with one or more programs that, when executed on one or more processors and/or controllers, perform the required functions. Various storage media may be fixed within a processor or controller or may be transportable, such that the one or more programs stored thereon can be loaded into a processor.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. If the term “adapted to” is used in the claims or description, it is noted that the term “adapted to” is intended to be equivalent to the term “configured to”. Any reference signs in the claims should not be construed as limiting the scope.

1. A system for generating a prediction for a first organ of a subject, the system comprising:
an input interface configured to obtain: first input data (x) representative of a detected property of the first organ (X) of the subject; and second input data (y) representative of a detected property of a second, different organ (Y) of the subject;
an data analysis component configured to determine a relationship between the first and second input data; and
a processor arrangement configured to:
evaluate the second input data with a digital twin model of the second organ to generate a prediction for the second organ; and
generate a prediction for the first organ based on the determined relationship and the prediction for the second organ.

2. The system of claim 1, wherein the data analysis component comprises:
a correlation component configured to determine a measure of correlation between the first (X) and second (Y) organs based on the first (x) and second (y) input data,
and wherein the data analysis component is configured to compare the determined measure of correlation between the first and second organs against a requirement and to determine a correlation function representative of a mapping between the first and second input data based on the comparison result.

3. The system of claim 2, wherein correlation component is configured to determine an inner product of the first and second input data.

4. The system of claim 2, wherein the input interface is configured to obtain third input data representative of a detected property of a third organ of the subject,
wherein the correlation component is further configured to determine a measure of correlation between the first and third organ based on the first and third input data,
and wherein the data analysis component is configured to determine the requirement based on the determined measure of correlation between the first and third organs.

5. The system of claim 4, wherein the data analysis component is configured to determine the requirement to be that the measure of correlation between the first and second organs must exceed the measure of correlation between the first and third organs, and
and wherein the data analysis component is configured to determine a correlation function representative of a mapping between the first and second input data responsive to the requirement being met.

6. The system of claim 1, wherein determining the relationship between the first and second input data comprises processing the first (x) and second (y) input data with at least one of: a parametric method; and a neural network.

7. The system of claim 1, wherein the processor arrangement is configured to generate a prediction for the first organ further based on a measure of variability of the first and second input data.

8. The system of claim 1, wherein the input interface is further configured to obtain supplementary input data representative of a detected property of the first organ of the subject at the future point in time,
and wherein the data analysis component is configured to determine an accuracy of the determined relationship based on the supplementary input data.

9. The system of claim 8, wherein the data analysis component is further configured to modify the determined relationship based on the supplementary input data.

10. The system of claim 1, further comprising at least one of:
a first set of one or more sensors (15A) configured to detect a property of the first organ (X) of the subject and to generate first input data (x) representative of the detected property of the first organ of the subject; and
a second set of one or more sensors (15B) configured to detect a property of the second organ (Y) of the subject and to generate second input data (y) representative of the detected property of the second organ of the subject.

11. The system of claim 11, wherein at least one of the first or second sets of one or more sensors comprises: a wearable sensor; a mobile phone; an digital imaging device; or a body implant.

12. A clinical decision support comprising a system for generating a prediction for a first organ of a subject according to claim 1.

13. A method for generating a prediction for a first organ of a subject, the method comprising:
obtaining: first input data (x) representative of a detected property of the first organ (X) of the subject; and second input data (y) representative of a detected property of a second, different organ of the subject;
determining a relationship between the first and second input data; and
evaluating the second input data with a digital twin of the second organ to generate a prediction for the second organ; and
generating a prediction for the first organ based on the determined relationship and the prediction for the second organ.

14. The method of claim 13, further comprising:
obtaining supplementary input data representative of a detected property of the first organ of the subject at the future point in time; and
determining an accuracy of the determined relationship based on the supplementary input data.

15. A computer program product comprising computer program code means which, when executed on a computing device having a processing system, cause the processing system to perform all of the steps of the method according to claim 13.
